(19) United States
(12) Patent Application Publication
(10) Pub. No.: US 2010/0071688 A1
(43) Pub. Date: Mar. 25, 2010

(54) Universal Self-Closing Metered Dose Inhaler Adaptor

(76) Inventor: Daniel Patrick DWYER, Raleigh, NC (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 12/234,887

(22) Filed: Sep. 22, 2008

(51) Int. Cl. A61M 11/00

(52) U.S. Cl. 128/200.18

(57) ABSTRACT

An embodiment in accordance with the present invention provides a universal, self-closing MDI adaptor for coupling an MDI to a breathing circuit without breaking the circuit. The present invention includes a self-closing inlet for coupling to an MDI, an inlet for coupling to an inspiratory limb of a breathing circuit, as well as an outlet for coupling the adaptor to the breathing circuit.

### FIELD OF THE INVENTION

The present invention relates generally to breathing circuits. More particularly, the present invention relates to a universal adaptor for coupling a metered dose inhaler to a breathing circuit.

### BACKGROUND OF THE INVENTION

Metered dose inhalers (“MDI”) are used to deliver a specific amount of a medication to a user's lungs in aerosolized form for treatment of medical conditions such as asthma and chronic obstructive pulmonary disease (“COPD”). An MDI includes a canister containing the medication and a propellant. The canister fits into an actuator, which has a mouthpiece and is used to dispense the medication from the canister. Traditionally, the user of the MDI places the mouthpiece of the actuator into the mouth, depresses the canister and breathes in to inhale the dispensed medication into the lungs. However, the medications delivered via an MDI may also be needed to treat patients who are unable to breathe without the assistance of a respirator. Therefore, a means to administer medication via an MDI to a patient breathing with the assistance of a respirator is essential.

One such means for administering medication via an MDI to a patient breathing with the assistance of a respirator is to break the sealed inspiratory limb of the breathing circuit. Once the sealed circuit is broken the MDI can be used to spray the drug directly into the circuit.

Another such method is to provide an adaptor coupled to the breathing circuit into which the MDI can be sprayed so that the breathing circuit need not be broken. Yet, in a clinical setting, a variety of medications delivered via MDI are needed to treat patients. These medications are manufactured by different companies and have a variety of different actuator configurations. The different actuators do not fit all within one single adaptor design. Therefore, it is necessary for patient care facilities to stock several different types of adaptors to accommodate all of the different actuator designs, or to remove the canister from the actuator altogether and use an alternate means of dispensing the medication.

Accordingly, it is desirable to provide an apparatus with a universal design for coupling an MDI to a breathing circuit, such that it can be used to coupled any MDI to the breathing circuit without breaking the circuit.

### SUMMARY OF THE INVENTION

The foregoing needs are met, to a great extent, by the present invention, wherein in one aspect an apparatus is provided that in some embodiments an adaptor is provided to couple any MDI to a breathing circuit without breaking the circuit.

In accordance with one aspect of the present invention, a universal metered dose inhaler adaptor for use with a breathing circuit includes a chamber having a wall defining an interior space. The wall of the chamber defines a first inlet opening for coupling the adaptor to an inspiratory limb of the breathing circuit. The wall of the chamber also defines an outlet opening for coupling the adaptor to the breathing circuit. The wall of the chamber further defines a second inlet opening, and the second inlet opening is configured to couple to a metered dose inhaler. The chamber also defines a first flow path. The first flow path is defined by the first inlet opening and the interior space of the chamber, such that gas flow is directed toward the outlet opening. Additionally, the chamber defines a second flow path for an aerosolized drug dispensed into the second inlet opening by a metered dose inhaler. The second flow path is separated from the first flow path by an angle α, such that the aerosolized drug mixes with the air from the inspiratory limb to be inhaled by a patient.

In accordance with another aspect of the present invention, the universal metered dose inhaler adaptor includes a spring-loaded, self-closing door to close off the second inlet opening. Alternatively, the universal metered dose inhaler adaptor can include a self-closing duckbill seal to close off the second inlet opening or a self-closing septum seal to close off the second inlet opening.

In accordance with yet another aspect of the universal metered dose inhaler adaptor the chamber can take the shape of a generally y-shaped structure. Additionally, the second inlet opening can be adjacent to the first inlet opening of the adaptor or alternately the second inlet opening can be adjacent to the outlet opening. The angle α between the first flow path and the second flow path can be between 0° and 45°.

In accordance with another aspect of the present invention, a universal metered dose inhaler adaptor for use with a breathing circuit includes a chamber having a wall defining a generally y-shaped interior space and having a proximal end and a distal end. The wall of the chamber defines a first inlet opening positioned at the distal end of the chamber for coupling the adaptor to an inspiratory limb of the breathing circuit. The wall of the chamber also defines an outlet opening defined by the wall of the chamber and positioned at the proximal end of the chamber for coupling the adaptor to the breathing circuit. The wall of the chamber further defines a second inlet opening defined by the wall of the chamber and positioned at the distal end of the chamber, adjacent to the first inlet opening. The second inlet opening is configured to couple to a metered dose inhaler. The chamber also defines a flow path for an aerosolized drug dispensed into the second inlet opening by the metered dose inhaler. The flow path is defined by the second inlet opening and the chamber, such that the aerosolized drug is sprayed toward the outlet opening and mixes with gas flow from the inspiratory limb to be breathed by a patient.

In accordance with still another aspect of the present invention, the universal metered dose inhaler adaptor includes a spring-loaded, self-closing door to close off the second inlet opening. Alternatively, the universal metered dose inhaler adaptor can include a self-closing duckbill seal to close off the second inlet opening or a self-closing septum seal to close off the second inlet opening. The universal metered dose inhaler adaptor can be formed from a plastic having anti-static properties, and the first inlet opening and the second inlet opening can be separated by an angle between 0° and 45°.

In accordance with another aspect of the present invention, the universal metered dose inhaler adaptor for use with a breathing circuit includes a chamber having a wall defining an interior space and having a proximal end and a distal end. The wall of the chamber defines a first inlet opening positioned at the distal end of the chamber for coupling the adaptor to an inspiratory limb of the breathing circuit. The wall of the chamber also defines an outlet opening positioned at the proximal end of the chamber for coupling the adaptor to the breathing circuit. The wall of the chamber further defines a second inlet opening defined by the wall of the chamber and positioned at the proximal end of the chamber, adjacent to the outlet opening. The second inlet opening is configured to couple to a metered dose inhaler. The chamber also defines a flow path for an aerosolized drug dispensed into the second inlet opening by the metered dose inhaler. The flow path is defined by the second inlet opening and the chamber, such that the aerosolized drug is sprayed away from the outlet opening and mixes with gas flow from the inspiratory limb to be breathed by the patient.

In accordance with another aspect of the present invention, the universal metered dose inhaler adaptor includes a spring-loaded, self-closing door to close off the second inlet opening. Alternatively, the universal metered dose inhaler adaptor includes a self-closing duckbill seal to close off the second inlet opening, or a self-closing septum seal to close off the second inlet opening. The universal metered dose inhaler adaptor can be formed from a plastic having anti-static properties and the first inlet opening and the second inlet opening can be separated by an angle between 0° and 45°.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 illustrates a universal, self-closing MDI adaptor inserted into a breathing circuit according to an embodiment of the invention.

FIG. 2 is an exploded view of the universal, self-closing MDI adaptor illustrated in FIG. 1.

FIG. 3 is a cross-sectional view of the universal, self-closing MDI adaptor illustrated in FIGS. 1 and 2.

FIG. 4 illustrates a universal, self-closing MDI adaptor inserted into a breathing circuit according to another embodiment of the invention.

FIG. 5 illustrates an exploded view of a universal, self-closing MDI adaptor inserted into a breathing circuit according to the embodiment of the invention illustrated in FIG. 4.

FIG. 6 is a cross sectional view of the universal, self-closing MDI adaptor illustrated in FIGS. 4 and 5, prior to the insertion of an MDI.

FIG. 7 is a cross sectional view of the universal, self-closing MDI adaptor illustrated in FIGS. 4, 5 and 6, after insertion of an MDI.

FIG. 8 illustrates a universal, self-closing MDI adaptor, inserted into a breathing circuit according to still another embodiment of the invention.

FIG. 9 illustrates a self-closing duckbill valve in accordance with an embodiment of the invention.

FIG. 10 illustrates a self-closing septum valve in accordance with an embodiment of the invention.

FIG. 11 illustrates a protective cap in accordance with an embodiment of the invention.

FIG. 12 illustrates an MDI adaptor system in accordance with yet another embodiment of the invention.

### DETAILED DESCRIPTION

The invention will now be described with reference to the drawing figures, in which like reference numerals refer to like parts throughout. An embodiment in accordance with the present invention provides a universal, self-closing MDI adaptor for coupling an MDI to a breathing circuit. The device allows the use of newer model MDI adaptors with integral counters in breathing circuits without having to break a circuit connection. The device also aids in the prevention of ventilator associated pneumonia (VAP), because it does not require that the circuit be broken to administer treatment using an MDI. The MDI adaptor includes a self-closing inlet for coupling to an MDI, an inlet for coupling to an inspiratory limb of a breathing circuit, as well as an outlet for coupling the adaptor to the breathing circuit.

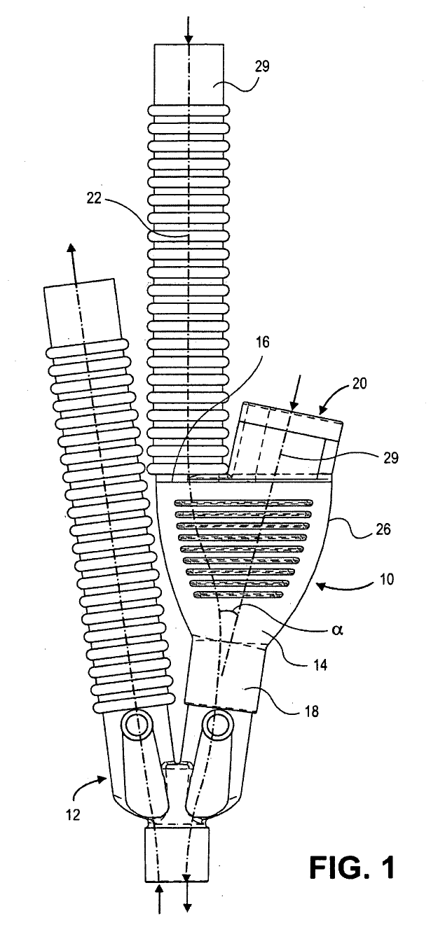

An embodiment of the present inventive apparatus is illustrated in FIG. 1. FIG. 1 illustrates a universal, self-closing MDI adaptor 10 for coupling an MDI to a breathing circuit 12. The adaptor 10 includes a chamber 14, a first inlet opening 16, an outlet opening 18, and a second inlet opening 20. The chamber 14 defines a first flow path 22, which can extend through inspiratory limb 29, and a second flow path 24 and defines a generally y-shaped structure. The chamber 14 also has a wall 26 defining an interior space 28 of the adaptor 10. Preferably, the adaptor is formed from a rigid elastomeric material having anti-static properties. However, any other suitable material may be used such as thermoplastic.

The first inlet opening 16 is defined by the wall 26 of the chamber 14 and is configured for coupling the adaptor 10 to an inspiratory limb 29 of the breathing circuit 12. The outlet opening 18 is also defined by the wall 26 of the chamber 14 and is configured to couple the adaptor 10 to the breathing circuit 12. Additionally, the second inlet opening 20 is defined by the wall 26 of the chamber 14 and is configured to couple to a metered dose inhaler (MDI), such that an aerosolized drug may be dispensed into the breathing circuit 12.

The first flow path 22 is defined by the first inlet opening 16 and the interior space of the chamber 28, such that gas can flow from the inspiratory limb 29 of the breathing circuit 12 and is directed toward the outlet opening 18. The second flow path 24 is defined by the second inlet opening 20 and extends into the chamber 14 and through the outlet opening 18. The second flow path 24 is configured, such that when an aerosolized drug is dispensed into the breathing circuit 12 the aerosolized drug mixes with the gas flowing from the inspiratory limb 29 through the first flow path 22 and can be inhaled by a patient. The second flow path 24 is separated from the first flow path by an angle α, as shown. The second inlet opening 20 can be positioned adjacent to the first inlet opening 16 such that the first flow path 22 is substantially parallel to the-second flow path 24. However, in one embodiment of the invention, the angle α between the first flow path 22 and the second flow path 24 can be anywhere from 0° to 45°.

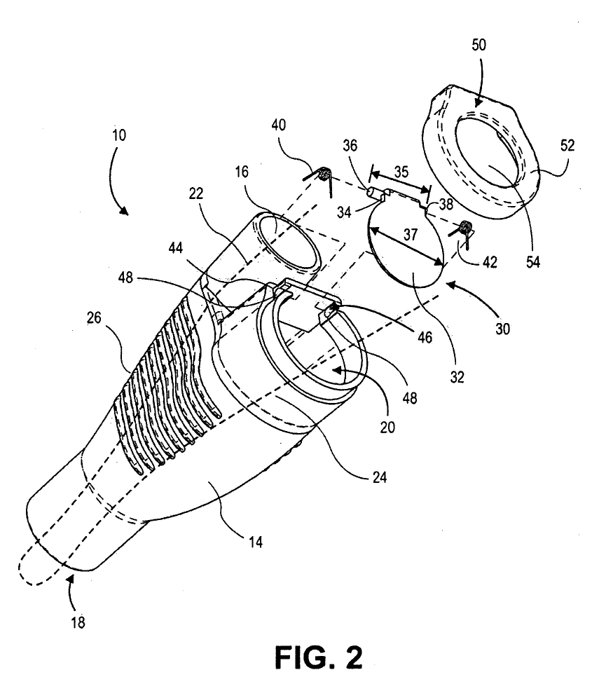

FIG. 2 is an exploded view that further illustrates the embodiment of the universal, self-closing MDI adaptor 10 illustrated in FIG. 1. The second inlet opening 20 includes a spring loaded door 30. As illustrated in FIG. 2, the spring loaded door 30 takes the form of a flat disc 32 having a generally cylindrical bar 34 positioned on one edge of the disc 32. The length 35 of the generally cylindrical bar 34, which functions as a hinge, is slightly longer than a diameter 37 of the flat disc 32. The generally cylindrical bar 34 includes a left end 36 and a right end 38, and springs 40, 42 are positioned around both the left end 36 and the right end 38. The left end 36 and the right end 38 fit into notches 44 and 46 defined by the wall 26 of the chamber 14. Notches 44 and 46 are slightly curved at their bottom edge 48, such that the generally cylindrical bar 34 sits rotatably within the notches 44 and 46. A cover 50 is positioned over the second inlet opening 20. The cover 50 keeps the spring loaded door 30 in place. A wall 52 of the cover defines a center opening 54 that exposes the spring loaded door 30 and accommodates a mouthpiece of an MDI.

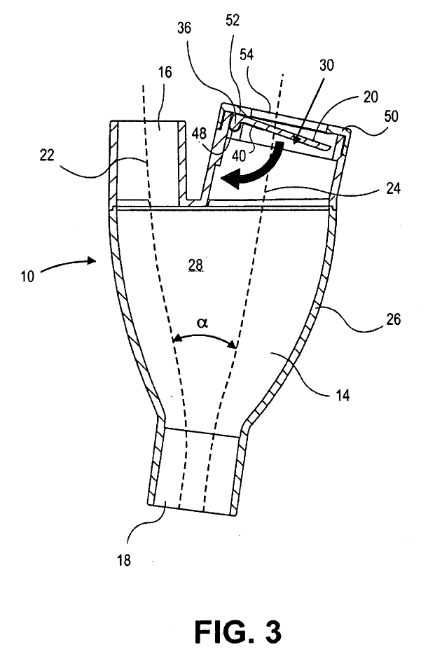

FIG. 3 is a cross sectional view of the universal, self-closing MDI adaptor illustrated in FIGS. I and 2. As illustrated in FIG. 3, the spring loaded door 30 swings into the interior space 28 of the chamber 14. When an MDI is inserted into the second inlet opening 20 through the center opening 54 of the cover 50, the spring loaded door 30 is pushed inward by the mouthpiece of the MDI. As, the generally cylindrical bar 34 rotates clockwise in notches 44 and 46, the springs 40 and 42 are compressed, and the door 30 rotates further. When the MDI is removed the springs 40 and 42 recoil and the door 30 rotates counter-clockwise and snaps closed.

The first flow path 22 is defined by the first inlet opening 16 and the interior space of the chamber 28, such that gas flow from the inspiratory limb 29 of the breathing circuit 12 is directed toward the outlet opening 18. The second flow path 24 is defined by the second inlet opening 20 and extends into the chamber 14 and through the outlet opening 18. The second flow path 24 is configured, such that when an aerosolized drug is dispensed into the breathing circuit 12 the aerosolized drug mixes with the gas flowing from the inspiratory limb through the first flow path 22 and can be inhaled by a patient. When the MDI is coupled to the second inlet opening 20 and the spring loaded door 30 is displaced, the MDI can be used to dispense the aerosolized drug. The aerosolized drug is dispensed by the MDI and a plume of the drug develops. The plume extends into the second flow path 24, and is well incorporated into the gas flowing through the inspiratory limb 29 and the first flow path 22 to be inhaled by the patient.

The second flow path 24 is separated from the first flow path by an angle α. The second inlet opening 20 can be positioned adjacent to the first inlet opening 16 such that the first flow path 22 is substantially parallel to the second flow path 24. However, the angle α between the first flow path 22 and the second flow path 24 can be anywhere from 0° to 45°. The first flow path 20 and the second flow path 22 merge at the outlet 18 such that the gas from the inspiratory limb 29 mixes with the plume of aerosolized drug dispensed by the MDI to be inhaled by the patient.

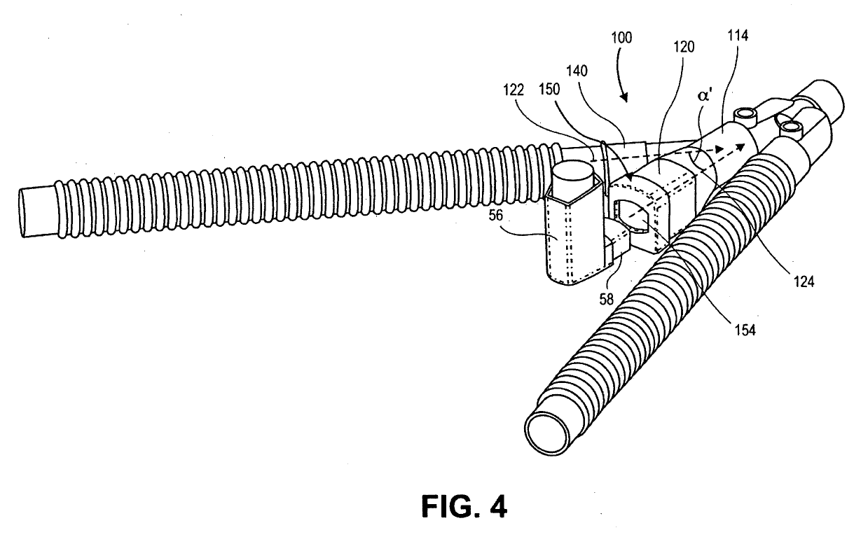

FIG. 4 illustrates a universal, self closing MDI adaptor 100 according to another embodiment of the invention. An MDI 56 having a mouthpiece 58 couples with the second inlet opening 120 of the adaptor 100. As shown in FIG. 4, a center opening 154 of a cover 150 can take a shape to couple with a shape of the mouthpiece 58 of numerous varieties of MDIs. A first inlet opening 116 and a second inlet opening 120 are positioned to form a generally y-shaped chamber 114. Therefore, a first flow path 122 and a second flow path 124 are separated by an angle α′ of 45°.

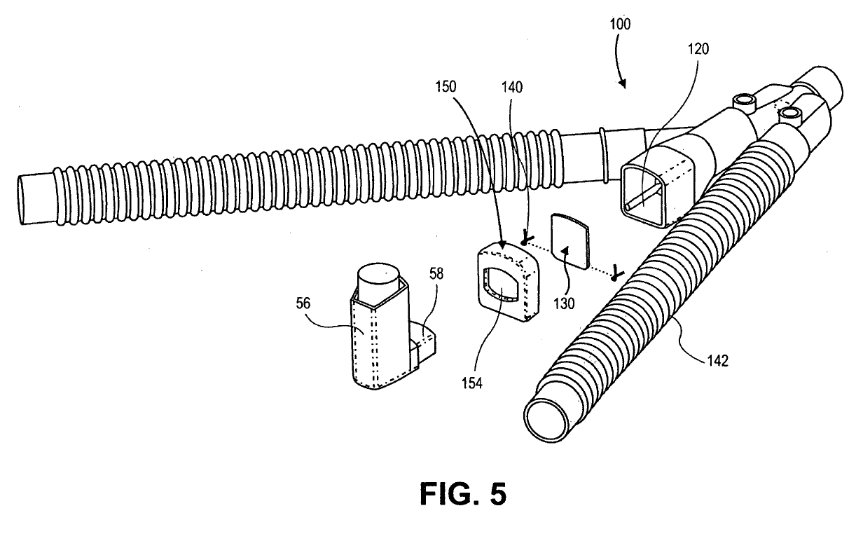

FIG. 5 is an exploded view that further illustrates the embodiment of the universal, self-closing MDI adaptor 100 illustrated in FIG. 4. The center opening 154 of the cover 150 is generally elliptical in shape having squared off ends in order to couple with the mouthpiece 58 of the MDI 56 or can take a shape that accommodates the mouthpieces of numerous MDIs. The center opening 154 of the cover 150 can take any shape necessary to couple with the MDI 56. The second inlet opening 120 and a spring loaded door 130 are generally square in shape however the second inlet opening 120 can take any shape necessary to couple to the MDI 56.

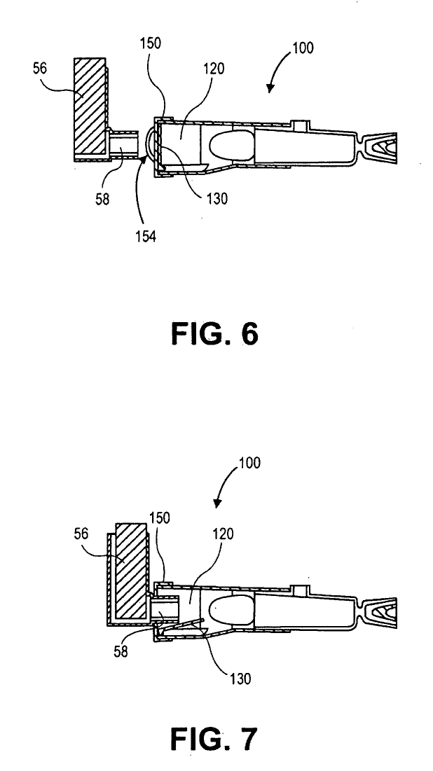

FIGS. 6 and 7 are cross sectional views of the universal, self-closing MDI adaptor illustrated in FIGS. 4 and 5. As illustrated in FIG. 6, the spring loaded door 130 remains closed when not in use, due to the tension in springs 140 and 142. As illustrated in FIG. 7, when the MDI 56 is inserted through the center opening 154 of the cover 150 and into the second inlet opening 120, the spring loaded door 130 rotates clockwise and the springs 140 and 142 are compressed to allow the spring loaded door to open.

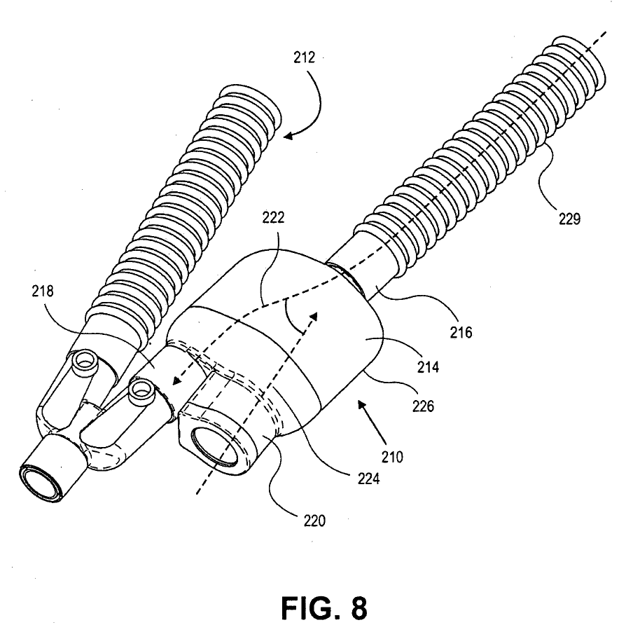

FIG. 8 illustrates a universal, self-closing MDI adaptor according to an embodiment of the invention inserted into a breathing circuit 212. The adaptor 210 includes a chamber 214, a first inlet opening 216, an outlet opening 218, and a second inlet opening 220. The chamber 14 defines a first flow path 222 and a second flow path 224. The chamber 214 also has a wall 226 defining an interior space 228 of the adaptor 210 and defines a generally y-shaped structure. Preferably, the adaptor is formed from a rigid elastomeric material having anti-static properties. However, any other suitable material may be used such as a thermoplastic.

The first inlet opening 216 is defined by the wall 226 of the chamber 214 and is configured for coupling the adaptor 210 to an inspiratory limb 229 of the breathing circuit 212. The outlet opening 218 is defined by the wall 226 of the chamber 214 and is configured to couple the adaptor 210 to the breathing circuit 212. Additionally, the second inlet opening 220 is defined by the wall 226 of the chamber 214 and is configured to couple to a metered dose inhaler (MDI), such that an aerosolized drug may be dispensed into the breathing circuit 212. The second inlet opening 220 is adjacent to the outlet opening 218, such that the structure is generally y-shaped.

The first flow path 222 is defined by the first inlet opening 216 and the interior space of the chamber 228, such that gas flow from the inspiratory limb 229 of the breathing circuit 212 is directed toward the outlet opening 218. The second flow path 224 is defined by the second inlet opening 220 and extends into the chamber 214, such that the aerosolized drug dispensed by the MDI is sprayed away from the outlet 218 and mixes with the gas flow through the first flow path 222 to be breathed by the patient. The second flow path 224 is separated from the first flow path by an angle β. The second inlet opening 220 can be positioned adjacent to the outlet 218 such that the first flow path 222 is parallel to the second flow path 224. However, the angle β between the first inlet opening 216 and the second inlet opening 220 can be anywhere from 0° to 45°.

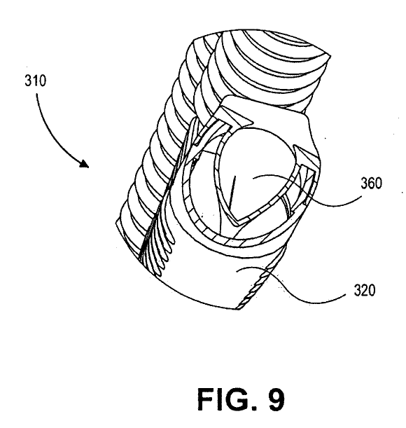

FIG. 9 illustrates a self-closing duckbill valve in accordance with an embodiment of the invention. The self-closing duckbill valve 360 is configured to couple to a second inlet opening 320 of an adaptor 310 and also to an MDI. The self-closing duckbill valve 360 is disposed within the second inlet opening 320 of the adaptor 310. A cover defining a center opening can be placed over the self-closing duckbill valve 360 in order to keep it in place within the second inlet opening 320. The self-closing duckbill valve 360 is formed from an elastomeric plastic or another similar material which allows the insertion of the mouthpiece of an MDI and returns to its original position after the mouthpiece of the MDI is removed.

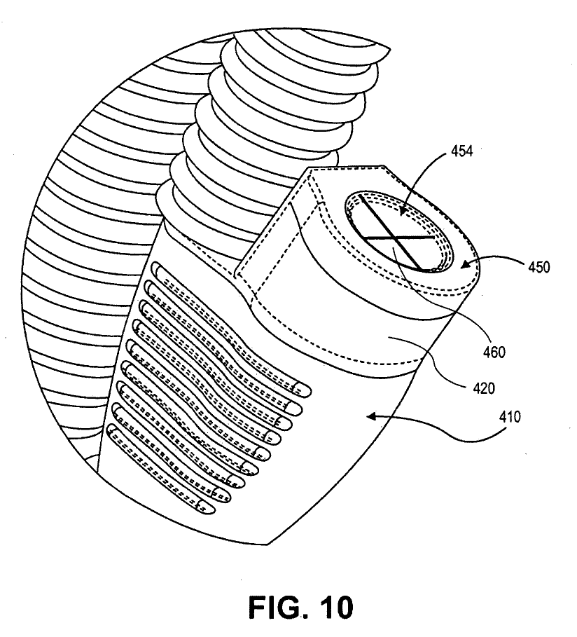

FIG. 10 illustrates a self-closing septum valve in accordance with an embodiment of the invention. The self-closing septum valve 460 is configured to couple to a second inlet opening 420 of an adaptor 410 and also to an MDI. The self-closing septum valve 460 is disposed within the second inlet opening 420 of the adaptor 410. A cover 450 defining a center opening 454 is placed over the self-closing septum valve 460 in order to keep it in place within the second inlet opening 460. The self-closing septum valve 460 is formed from an elastomeric plastic or another similar material which allows the insertion of the mouthpiece of an MDI and returns to its original position after the mouthpiece MDI is removed.

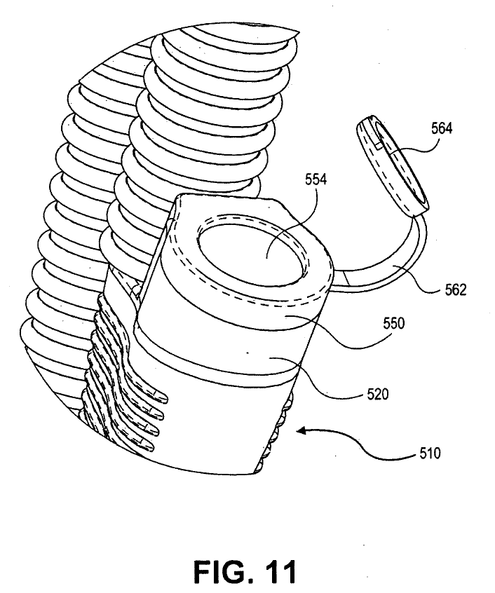

FIG. 11 illustrates a protective cap in accordance with an embodiment of the invention. A tether 562 couples a protective cap 564 to a cover 550. The protective cap 564 is shaped to fit within a center opening 554 defined by the cover 550. The center opening 554 is in fluid communication with a second inlet opening 520 defined by a chamber 514 of the MDI adaptor 510. The protective cap can be placed within the center opening 554 of the cover 550 to prevent any environmental debris, dust, or other contaminants to enter the adaptor 510 when it is not in use. The protective cap 564 can be formed from the same rigid elastomeric material as the adaptor 510, or can be formed of any other suitable material.

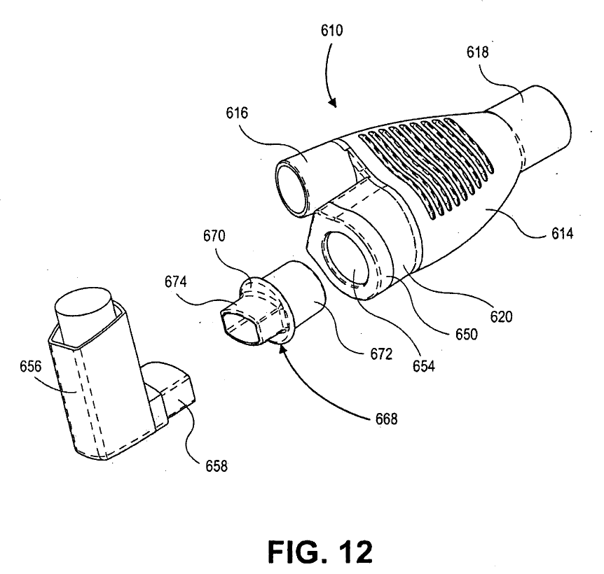

FIG. 12 illustrates a coupler for an MDI adaptor 610 in accordance with an embodiment of the invention. The MDI adaptor includes a chamber 614, a first inlet opening 616, and outlet opening 618, and a second inlet opening.620. A coupler 668 can be used to couple an MDI 656 having a mouthpiece 658 with a shape different from that of the opening 654 defined by the cover 650 to the adaptor 610. As illustrated in FIG. 12, the opening 654 of the cover 650 is generally circular in shape. A wall 670 of the coupler 668, therefore, defines a generally cylindrical end 672 to be coupled to the opening 664. The wall of the coupler 668 defines a second end 674 shaped such that it will couple to the mouthpiece 658 of the MDI 656. The generally cylindrical end 672 of the coupler 668 can be placed into the opening 654 to displace the spring loaded door or seal used to cover the second inlet opening 620. Once the coupler 668 is inserted into the adaptor 610, the MDI can be coupled to the second end 674 and dispensed into the adaptor 610. The coupler 668 may be formed from a rigid elastomeric material or any other suitable material such as a thermoplastic.

The device described above, therefore, allows the use of newer model MDI adaptors with integral counters in breathing circuits without having to break a circuit connection. The device also aids in the prevention of VAP, because it does not require that the circuit be broken to administer treatment using an MDI.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A universal metered dose inhaler adaptor for use with a breathing circuit comprising:
a chamber having a wall defining an interior space;
a first inlet opening defined by the wall of the chamber for coupling the adaptor to an inspiratory limb of the breathing circuit;
an outlet opening defined by the wall of the chamber for coupling the adaptor to the breathing circuit;
a second inlet opening defined by the wall of the chamber, wherein the second inlet opening is configured to couple to a metered dose inhaler;
the chamber defining a first flow path, wherein the first flow path is defined by the first inlet opening and the interior space of the chamber, such that gas flow is directed toward the outlet opening; and
the chamber defining a second flow path for an aerosolized drug dispensed into the second inlet opening by a metered dose inhaler, wherein the second flow path is separated from the first flow path by an angle α, such that the aerosolized drug mixes with the air from the inspiratory limb to be inhaled by a patient.

2. The universal metered dose inhaler adaptor of claim 1 further comprising a spring-loaded, self-closing door to close off the second inlet opening.

3. The universal metered dose inhaler adaptor of claim 1 further comprising a self-closing duckbill seal to close off the second inlet opening.

4. The universal metered dose inhaler adaptor of claim 1 further comprising a self-closing septum seal to close off the second inlet opening.

5. The universal metered dose inhaler adaptor of claim 1, wherein the chamber comprises a generally y-shaped structure.

6. The universal metered dose inhaler adaptor of claim 1, wherein the second inlet opening is adjacent to the first inlet opening.

7. The universal metered dose inhaler adaptor of claim 1, wherein the second inlet opening is adjacent to the outlet opening.

8. The universal metered dose inhaler adaptor of claim 1, wherein the angle α is between 0° and 45°.

9. A universal metered dose inhaler adaptor for use with a breathing circuit comprising:
a chamber having a wall defining a generally y-shaped interior space and having a proximal end and a distal end;
a first inlet opening defined by the wall of the chamber and positioned at the distal end of the chamber for coupling the adaptor to an inspiratory limb of the breathing circuit;
an outlet opening defined by the wall of the chamber and positioned at the proximal end of the chamber for coupling the adaptor to the breathing circuit;
a second inlet opening defined by the wall of the chamber and positioned at the distal end of the chamber, adjacent to the first inlet opening, wherein the second inlet opening is configured to couple to a metered dose inhaler; and
the chamber defining a flow path for an aerosolized drug dispensed into the second inlet opening by the metered dose inhaler, wherein the flow path is defined by the second inlet opening and the chamber, such that the aerosolized drug is sprayed toward the outlet opening and mixes with gas flow from the inspiratory limb to be breathed by a patient.

10. The universal metered dose inhaler adaptor of claim 9 further comprising a spring-loaded, self-closing door to close off the second inlet opening.

11. The universal metered dose inhaler adaptor of claim 9 further comprising a self-closing duckbill seal to close off the second inlet opening.

12. The universal metered dose inhaler adaptor of claim 9 further comprising a self-closing septum seal to close off the second inlet opening.

13. The universal metered dose inhaler adaptor of claim 9, wherein the adaptor comprises a plastic having anti-static properties.

14. The universal metered dose inhaler adaptor of claim 9 wherein the first inlet opening and the second inlet opening are separated by an angle between 0° and 45°.

15. A universal metered dose inhaler adaptor for use with a breathing circuit comprising:
a chamber having a wall defining an interior space and having a proximal end and a distal end;
a first inlet opening defined by the wall of the chamber and positioned at the distal end of the chamber for coupling the adaptor to an inspiratory limb of the breathing circuit;
an outlet opening defined by the wall of the chamber and positioned at the proximal end of the chamber for coupling the adaptor to the breathing circuit;
a second inlet opening defined by the wall of the chamber and positioned at the proximal end of the chamber, adjacent to the outlet opening, wherein the second inlet opening is configured to couple to a metered dose inhaler;
the chamber defining a flow path for an aerosolized drug dispensed into the second inlet opening by the metered dose inhaler, wherein the flow path is defined by the second inlet opening and the chamber, such that the aerosolized drug is sprayed away from the outlet opening and mixes with gas flow from the inspiratory limb to be breathed by the patient.

16. The universal metered dose inhaler adaptor of claim 15 further comprising a spring-loaded, self-closing door to close off the second inlet opening.

17. The universal metered dose inhaler adaptor of claim 15 further comprising a self-closing duckbill seal to close off the second inlet opening.

18. The universal metered dose inhaler adaptor of claim 15 further comprising a self-closing septum seal to close off the second inlet opening.

19. The universal metered dose inhaler adaptor of claim 15, wherein the adaptor comprises a plastic having anti-static properties.

20. The universal metered dose inhaler adaptor of claim 15 wherein the first inlet opening and the second inlet opening are separated by an angle between 0° and 45°.
